(19) United States
(12) Patent Application Publication
(10) Pub. No.: US 2008/0139899 A1
(43) Pub. Date: Jun. 12, 2008

(54) Remote Monitoring System For Alzheimer Patients

(76) Inventors: Menachem Student, Gat Rimon (IL); Nir Karasikov, Haifa (IL); David Tamir, Haifa (IL); Revital Naveh Bar - On, Tel Aviv (IL)

Correspondence Address: FLEIT KAIN GIBBONS GUTMAN BONGINI & BIANCO, 21355 EAST DIXIE HIGHWAY, SUITE 115, MIAMI, FL, 33180, US

(21) Appl. No.: 11/913,439

(22) PCT Filed: May 4, 2006
(86) PCT No.: PCT/IL2006/000531

§ 371 Date: Nov. 2, 2007

Related U.S. Application Data
(60) Provisional application No. 60/677,405, filed on May 4, 2005.

(51) Int. Cl. A61B 5/00

(52) U.S. Cl. 600/301

(57) ABSTRACT

Disclosed is a device which helps caregivers of demented patients, such as persons suffering from Alzheimer's disease, monitor and safeguard the wellbeing of the patients. The device, which is attached to the wrist or the leg of the patients, monitors the movement and behavior of the patient and informs the caregivers whenever unexpected or alarming incidences occur. The device may identify if the patient has fallen, wondered off or is inactive for long periods of time. The device records and learns the patterns of daily behavior of the patient in relation to which it may identify unexpected occurrences. The device is equipped with sensors which enable it to monitor the location, behavior and condition of the patient. Additionally, the device may be programmed to identify particular behaviors as alarming. Selectively activating its sensors and communication interface, the device is especially equipped to operate on low battery consumption.

### FIELD OF THE INVENTION

The present invention relates in general to devices which aid caregivers of demented patients, such as Alzheimer's disease patients, monitor the activity of the patients; more particularly it relates to devices which track the activity of demented patients, identifies irregular and possibly alarming activity and alarm the caregivers of the patients when a state of emergency occurs.

### BACKGROUND

Caregivers of patient suffering from Alzheimer's disease and related dementia diseases face numerous problems. In particular such patients need constant monitoring to ensure their wellbeing and that they do not harm themselves. Additionally, demented and Alzheimer's disease patient occasionally have a tendency to wonder off and are than needed to be located by their caregivers. According to research, 70 percent of all Alzheimer's patients wander from their assisted-living facilities and homes at least once. 3 of 4 cases, who wandered once, will wander more than once. If not found within 24 hours, their chances of dying are greater than 50 percent. Identifying that a patient is lost and locating their whereabouts is therefore of an at most urgency.

Known in the art are a wide variety of devices which may be carried on the body of a patient and monitor the activity and location of the patient. US Patent Application No. 20020193091, for instance, is one such device. The device includes a combination of sensors which constantly monitor the condition and location of the patient such as a continuous accelerometer switches. A remote monitoring system shows the exact location of the individual and displays the proper medical prompts according to patient's history or recorded instructions. Whenever an emergency occurs, or if the patient is lost, the system finds the exact location of the patient and informs the caregiver.

One of the major shortcomings of devices such as disclosed in US Patent Application No. 20020193091 is that in order to operate efficiently they rely on relatively high levels of energy consumption which demand using stronger and bigger batteries or frequent battery replacement. Addressing this problem U.S. Pat. No. 5,461,365 describes a personal alarm system including a monitoring base station and one or more remote sensing units in two-way radio communication. The remote units transmit at selectable power levels. In the absence of an emergency, a remote unit transmits at a power-conserving low power level. Received field strength is measured to determine whether a remote unit has moved beyond a predetermined distance from the base station. If the distance is exceeded, the remote unit transmits at a higher power level. The remote unit includes sensors for common hazards including water emersion, smoke, excessive heat, excessive carbon monoxide concentration, and electrical shock.

However, to accurately identify a state of emergency the information received from these sensors may prove to be insufficient. For instance, the device should be able to distinguish between a normal walk taken by the patient and when the patient has wondered off and is lost. Similarly, the device should be able to identify dangerous bodily conditions which need immediate attention, such as when the patient has fallen, passed out or is in convulsions. For this purpose the proposed solution should include means for identifying behaviors and physical conditions of the patient which deviate from the normal and routine behavior of the patient. Additionally, the device should operate using minimal power consumption to ensure that the batteries last for long periods of time. Preferably, the device should include chargeable sources of energy and a charge generator.

### SUMMARY

Disclosed is a personal monitoring device attached to the body of the user, for alerting when abnormal behavior or condition of the monitored person is detected. The device is comprised of at least one location sensor for periodically identifying the location of the monitored person, at least one activity sensor for periodically identifying the movement of the monitored person and at least one physiological sensor for periodically measuring bodily functionality. Additionally, the device includes a wireless communication transmitter for conveying at least part of the collected data to a remote terminal, wherein the data to be transmitted is determined in accordance with predefined rules. Also included is a power source and a main processing unit device for collecting all information from the sensors, detecting abnormal behavior and managing the activation of the sensors and the transmitter for optimal power consumption operation, each sensor is activated for a minimal time period. The activation management of the sensors is based on user daily behavior pattern and analysis of detected abnormal behavior.

The device may also include an analyzing module for processing all collected data and determining the data to be transmitted. The user daily behavior pattern is determined by a learning phase module of the device.

The device may further comprise a beacon to be activated when there is no communication with monitored person. Upon activation the beacon repeatedly transmits signal to be identified by receivers of users trying to locate the monitored person. The transmitted signal may include one of the following: radio frequency, infrared, light, color, sound, ultrasound and odor. The location sensor may be a GPS sensor or a GSM component. The activity sensor may be one of the following: movement sensor, acceleration sensor or magnet sensor. The physiological sensor may be one of the following: heartbeat rate sensor, blood pressure sensor, blood-oxygen levels sensor, blood-sugar levers sensor or temperature sensor. Also included is a medication activator which is controlled by the main processing unit, wherein the activation is based on collected data from the sensors and known behavior patterns.

The device also includes a charge generator, wherein the power source is chargeable. The power charge generator may be one of the following: a solar cell, a RF energy converter, a piezoelectric element converting motion, or pressure into energy, a micro wave detector or a pyro electric device converting temperature changes to electric power.

The main processing unit includes a pattern behavior recognition module for detecting abnormal behavior. The device is attached to the monitored person hand or leg using a bracelet where the bracelet is lockable to the hand or the leg. The accelerometer of the device is activated periodically in accordance with the individual behavior pattern.

Also disclosed is a method for alerting when abnormal behavior or condition of the monitored person is detected using a personal monitoring device which is attached to the body of the user. The method includes the steps of periodically identifying the location of the monitored person by a dedicated sensor of the personal device, periodically identifying the movement of the monitored person by a dedicated sensor of the personal device and periodically measuring bodily functionality by a dedicated sensor of the personal device. The method also includes the steps of conveying at least part of the collected data to a remote terminal by transmitter of the sensor, wherein the data to be transmitted is determined in accordance with predefined rules, collecting all information from the sensors and detecting abnormal behavior in accordance with known daily behavior pattern. The method also includes the step of managing the activation of the sensors and transmitter for optimal power consumption operation, wherein each sensor is activated for a minimal time period, and the activation management of the sensors is based on user daily behavior pattern and analysis of detected abnormal behavior.

Additionally, the method may include the step of performing pattern behavior recognition for detecting abnormal behavior. The sensor may include a medication activator, further comprising the step of controlling the activator operation in accordance with collected data from the sensors and known behavior pattern.

### BRIEF DESCRIPTION OF THE DRAWINGS

These and further features and advantages of the invention will become more clearly understood in the light of the ensuing description of a preferred embodiment thereof, given by way of example, with reference to the accompanying drawing, wherein—

FIG. 1 is a block diagram illustrating the principal components of the disclosed device in accordance with the preferred embodiments of the present invention.

### DETAILED DESCRIPTION OF THE PREFERRED EMBODIMENTS

The present invention is a new and innovative device which helps caregivers of demented patients, such as persons suffering from Alzheimer's disease, monitor and safeguard the wellbeing of the patients. The device, which is attached to the wrist or the leg of the patients and may be locked to their wrist, monitors the movement and behavior of the patient and informs the caregivers whenever unexpected or alarming incidences occur. For instance, the device may identify if the patient has fallen, wondered off or is inactive for long periods of time. According to the preferred embodiments of the present invention the device records and learns the patterns of daily behavior of the patient in relation to which it may identify unexpected occurrences. For this purpose the device is equipped with sensors, which enable the device to monitor the location, behavior and condition of the patient. Additionally, the device may be programmed to identify particular behaviors as alarming, such as that of the patient leaving a designated area. Selectively activating its sensors and communication interface, the device is especially equipped to operate on low battery consumption.

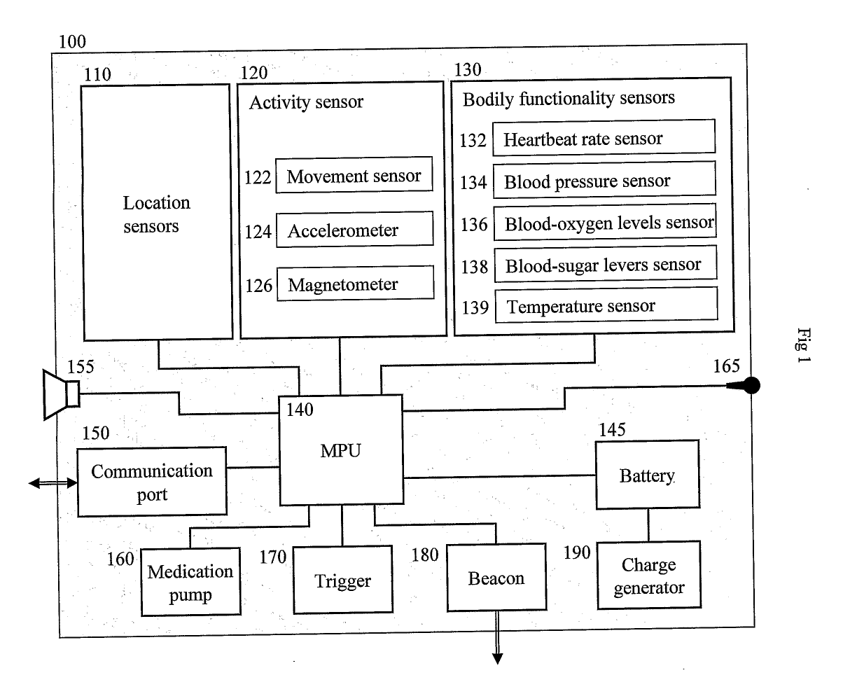

FIG. 1 is a block diagram illustrating the principal components of the present invention according to the preferred embodiments. According to the preferred embodiments device 100 is comprised of three types of sensors. The first sensor type is locating sensor 110. Locating sensor 110 may operate using different locating technologies known to persons who are skilled in the art. For instance, locating sensor 110 may be a Global Positioning System (GPS) sensor, or a cellular communication component, such as a Global System for Mobile Communications (GSM) component, which may connect to a local cellular network through which its location is determined. It could also be based on double integration of the signal of an accelerometer 124, as and described below. In addition to locating sensor 110 device 100 may also include beacon 180. Beacon 180 may be activated when device 100 identifies that the patient is lost. When activated, beacon 180 repeatedly transmits the signal which may aid the caregivers of the patient pinpoint the exact location of the patient. Beacon 180 may transmit a signal in radio frequency (RF), infrared (IR), light, color, sound, ultrasound or odor provided that the search is conducted using trained dogs. Additionally, a micro air vehicle (MAV), unattended air vehicle (UAV) may be used to facilitate the search.

The second type of sensors is activity sensor 120. According to the data recorded by activity sensor 120, which monitors the movement of the device, the movement of the body of the patient carrying the device may be extrapolated by a double integration. According to one embodiment of the present invention activity sensor 120 combines several motion sensing abilities. For instance, activity sensor 120 may includes movement sensor 122 which tracks any simple movement made by the patient. Movement sensor may provide indications for the level of activity of the patient. Activity sensor 120 may also be comprised of accelerometer 124, which identifies when the patient is riding in a vehicle or when short rapid movements are made by the patient, such as when falling down. Additionally, activity sensor may also include magnetometer 126, which may measure the proximity of the patient to the ground. Magnetometer 126 is a three-directional magnetometer sensing the earth magnetic field. Magnetometer 126 aids in identifying whenever the patient has fallen and is lying on the ground, by identifying that the stronger axis is different for extended period of time. Combining data gathered by activity sensors 120, device 100 may analyze the behavior of the patient and compare it with the established daily pattern of behavior. Thus, the device may identify when too much or too little activity is measured, or when particular behavior patterns appear in unexpected times of the day, such as high levels of activity during the night.

The third type of sensors is bodily functionality sensors 130. Bodily functionality sensors 130 may include a wide variety of sensors which may provide indications as for the status of the body of the patient. For instance, bodily functionality sensors may include sensors measuring the pulse of the patient 132, blood pressure 134, blood-oxygen levels 136, blood-sugar levers 138 or temperature 139. The preferred embodiments of the present invention may include any other type of sensor which can provide essential indications as for the status of the body of the patient, using the open architecture structure of the device. Even more so, the type of sensors integrated into any device may be especially selected to suit the needs of the patient. Thus, for a diabetic patient the blood-sugar sensor 138 may be in need while for a patient suffering from blood-pressure anomalies the heartbeat rates sensor 132 and blood pressure sensor 134 may prove to be more essential.

Additional sensors may be added according to the particular needs of the patient. For instance, smoke sensors may give an additional safety mechanism to dementia patients. All data gathered by the sensors is collected and processed by the main processing unit 140. Device (MPU) 100 is powered by mobile power source 145, which may be chargeable through charge generator 190. Charge generator 190 may operate using a piezoelectric element converting motion or pressure into electric energy. A pyro-electric device could convert temperature changes into electric energy. Alternatively, generator 190 may operate using solar, RF, microwave or any other type of energy which may provide recharging abilities. The main processing unit 140 also controls the activation of the different sensors to minimize the power consumption of the device according to consideration which are described below. To reduce the needed computation resources all data may be sent through communication port 150, processed by a remote computation center and sent back to main processing unit 140.

Device may also include an emergency button. When the emergency button is pressed device 100 may communicate with a 911 emergency, a medical emergency or with the caregivers of the patient through communication port 150. Vocal two-way communication may be established through the device using microphone 165 and speaker 155.

According to the preferred embodiments of the present invention the normal operation of the device is enabled by a preliminary learning phase. In the learning phase, which may be enabled by artificial intelligence, the device is attached to the body of the patient and monitors his or her behavior. The device records the daily behavior of the patient and recognizes reoccurring patterns. For instance, the device may identify when the patient usually sleeps and for how long, and the daily routine of the different levels of activity of the patient. Additionally, the device also learns where the patient usually resides and the routes that he or she usually takes. The device records the location of the main residence of the patient and the location of the places where the patient visits frequently, such as the shops, the park or the clinic. An algorithm then monitors deviation from the normal behavior patterns. As mentioned above, the device may also be programmed to identify when the patient moves outside a predefined area. For instance, the device may alert the caregivers of a patient when the device identifies that the patient is leaving his or her house during the night.

The period of time which is dedicated to establishing the daily behavior of the patient may vary according to the different parameters. Patients who follow a strict routine may need shorter learning phases than patients whose behavior and activity are much more diverse. Additionally, particular conditions may be programmed as alarming by the caregivers or the doctors of the patient. For instance, when predefined bodily conditions, such as blood pressure dropping below a particular threshold or when increased levels of activity are identified by the device.

Having established the daily pattern of activity of the patient the device is switched to normal operation mode. Relying on the recorded pattern of daily activity the device does not have to operate the fill range of sensors at its disposal to ensure the wellbeing of the patient. The device may selectively and periodically operate the different sensors and compare their readings to the values which are expected from the established pattern. This feature enables the device to be particularly economic in its use of battery power. For this purpose sensors which demand high levels of energy to operate may be activated only in predetermined time periods or when other sensors or algorithms show indications of abnormal circumstances. Whenever alarming circumstances are detected by the device trigger 170 activates the full facility of the device and commences the emergency protocol.

According to the medical and behavioral profile of the patient, particular sensors may be programmed to operate more frequently than others. For instance, if the patient suffers from a heart condition the sensors monitoring heartbeat rate and blood pressure levels may operate more frequently than they would do with other types of patients. Similarly, demented patients which have a tendency to wonder off would require the location sensors to operate in short intervals to ensure that the patient is not lost.

Whenever alarming circumstances are identified by the device, the caregivers of the patient are notified using one of several methods. The caregivers may be contacted through their mobile phone, wired phone, personal digital assistant (PDA) device or through a dedicated device. The device may send a prerecorded vocal or text message or simply sound an alarm. The prerecorded messages may include information about the type of condition which is identified by the device. For instance, the message may say that the patient has wondered off, that there is a chance that the patient has fainted, that the measured blood pressure is found to diverge from normal levels or that he or she did not get up from bed in the morning. When the patient is identified as having wondered off the device may also transmit an image of the patient to the caregivers and the rescuing teams to aid in identifying the patient. When operating the device in an institution, all active devices in the institution may be centrally monitored. Thus, from a central location all patients who carry the device may be located and their condition reported.

According to an additional embodiment of the present invention, the device may communicate with a camera located at the exit of the facility where the patients reside. Whenever a patient wearing the device leaves the premises, the device activates the camera and an updated picture of the patient is stored. Thus, if the patient wonders off and needs to be located, the search team may extract this picture and would therefore have the exact and updated details of the clothes and the looks of patient. This visual information may also be fed into other systems, such as metropolitan surveillance systems, to aid with the search.

In addition to informing the caregivers about the condition of the patient, the device may also sound a message for the patient. For instance, the device may play a vocal message reminding the patient to take certain medication. According to an additional embodiment of the present invention the device may be connected to the medication infusion pump of the patient, such as an insulin pump, and control its operation whenever predefined bodily conditions are recorded through medication activator 160. Any combination of these modes of operation may be programmed to be activated whenever predefined circumstances are met.

While the above description contains many specifications, these should not be construed as limitations on the scope of the invention, but rather as exemplifications of the preferred embodiments. Those skilled in the art will envision other possible variations that are within its scope. Accordingly, the scope of the invention should be determined not by the embodiment illustrated, but by the appended claims and their legal equivalents.

What is claimed is:

1. A personal monitoring device attached to the body of the user, for alerting when abnormal behavior or condition of the monitored person is detected, said device comprised of:
at least one location sensor for periodically identifying the location of the monitored person;
at least one activity sensor for periodically identifying the movement of the monitored person;
at least one physiological sensor for periodically measuring bodily functionality;
a wireless communication transmitter for conveying at least part of the collected data to a remote terminal, wherein the data to be transmitted is determined in accordance with predefined rules;
a main processing unit device for collecting all information from the sensors, detecting abnormal behavior and managing the activation of the sensors and the transmitter for optimal power consumption operation, each sensor is activated for a minimal time period, wherein the activation management of the sensors is based on user daily behavior pattern and analysis of detected abnormal behavior;
a power source.

2. The device of claim 1 further comprising an analyzing module for processing all collected data and determining the data to be transmitted.

3. The device of claim 1 wherein the user daily behavior pattern is determined by a learning phase module of the device.

4. The device of claim 1 further comprising a beacon to be activated when there is no communication with monitored person, wherein upon activation the beacon repeatedly transmits signal to be identified by receivers of users trying to locate the monitored person.

5. The device of claim 3 wherein the transmitted signal is one of the following: radio frequency, infrared, light, color, sound, ultrasound, odor.

6. The device of claim 1 wherein the location sensor is one of the following: GPS sensor, GSM component.

7. The device of claim 1 wherein the activity sensor is at least one of the following: movement sensor, acceleration sensor, magnet sensor.

8. The device of claim 1 wherein the physiological sensor is at least one of the following: heartbeat rate sensor, blood pressure sensor, blood-oxygen levels sensor, blood-sugar levers sensor, temperature sensor.

9. The device of claim 1 further comprising a medication activator which is controlled by the main processing unit, wherein the activation is based on collected data from the sensors and known behavior patterns.

10. The device of claim 1 further comprising a charge generator, wherein the power source is chargeable.

11. The device of claim 10 wherein the power charge generator is one of the following: a solar cell, a RF energy converter, a piezoelectric element converting motion, or pressure into energy, a micro wave detector or a pyro electric device converting temperature changes to electric power.

12. The device of claim 1 wherein the main processing unit includes a pattern behavior recognition module for detecting abnormal behavior.

13. The device of claim 1 wherein the device is attached to the monitored person hand using a bracelet where the bracelet is lockable to the hand.

14. The device of claim 1 wherein the device is attached to the leg.

15. The device of claim 1 wherein the accelerometer is activated periodically in accordance with the individual behavior pattern.

16. A method for alerting when abnormal behavior or condition of the monitored person is detected using a personal monitoring device which is attached to the body of the user, said method comprising the steps of:
periodically identifying the location of the monitored person by a dedicated sensor of the personal device;
periodically identifying the movement of the monitored person by a dedicated sensor of the personal device;
periodically measuring bodily functionality by a dedicated sensor of the personal device;
conveying at least part of the collected data to a remote terminal by transmitter of the sensor, wherein the data to be transmitted is determined in accordance with predefined rules;
collecting all information from the sensors;
detecting abnormal behavior in accordance with known daily behavior pattern; and
managing the activation of the sensors and transmitter for optimal power consumption operation, wherein each sensor is activated for a minimal time period, and the activation management of the sensors is based on user daily behavior pattern and analysis of detected abnormal behavior.

17. The method of claim 16 further comprising the step of performing pattern behavior recognition for detecting abnormal behavior.

18. The method of claim 16 wherein the sensor includes a medication activator, further comprising the step of controlling the activator operation in accordance with collected data from the sensors and known behavior pattern.
